(19) United States
(12) Patent Application Publication
(10) Pub. No.: US 2025/0257312 A1
(43) Pub. Date: Aug. 14, 2025

(54) CONTAMINATION CONTROL WHEN GROWING GREEN ALGAE

(71) Applicant: Edward Brian HAMRICK, Sunny Isles Beach, FL (US)

(72) Inventor: Edward Brian HAMRICK, Sunny Isles Beach, FL (US)

(21) Appl. No.: 18/640,396

(22) Filed: Apr. 19, 2024

Related U.S. Application Data
(60) Provisional application No. 63/551,331, filed on Feb. 8, 2024.

(51) Int. Cl. C12N 1/12; C12M 1/02; C12M 1/34

(52) U.S. Cl. CPC C12N 1/12; C12M 41/18; C12M 41/26; C12M 41/32

(57) ABSTRACT

A method for bacterial contamination control of heterotrophic growth of green algae is provided. Bacterial contamination is controlled by using urea as the primary nitrogen source while simultaneously limiting the amount of nickel available to contaminating bacteria. Bacteria require nickel as a cofactor for urease enzymes in order to use urea for growth while green algae do not require nickel as a cofactor for any enzymes. Green algae use biotin as a cofactor to use urea and are biotin prototrophic. Nickel is limited by using titanium in plate heat exchangers instead of stainless steel. The preferred green algae are Chlorella sorokiniana, Chlorella vulgaris, and Auxenochlorella protothecoides.

### PRIORITY DATA

This patent application is a non-provisional application claiming priority to U.S. Provisional Patent App. No. 63/551,331, filed on Feb. 8, 2024, which is hereby incorporated by reference herein for all purposes.

### FIELD OF THE INVENTION

The present invention pertains to growth of microorganisms. More specifically, this invention pertains to a method for contamination control when growing green algae, which is the common term for algae in the Chlorophyta division of Eukaryota.

### BACKGROUND OF THE INVENTION

Green algae are a division of Eukaryota called Chlorophyta (also referred to as chlorophytes). The fastest growth of green algae uses heterotrophic growth, which is growth in the dark on combinations of sugars, acetate and glycerol. The fastest-growing green algae when growing on glucose in the dark are Chlorella sorokiniana, Chlorella vulgaris, andAuxenochlorella protothecoides, with Chlorella sorokiniana growing the fastest, having a doubling time of about nine hours.

Green algae have been grown for millennia as food. Green algae contain a significant amount of well-balanced protein, Omega-3 fatty acids such as alpha-linolenic acid (ALA), docosahexaenoic acid (DHA) and eicosapentaenoic acid (EPA) as well as other valuable phytochemicals such as lutein.

Industrial-scale growth of green algae commonly uses what herein is referred to as a “fermenter”, which is an enclosed space without light that uses fed-batch addition of sugars and nutrients, is aerated with oxygen and kept at an optimal growth temperature. This growth is herein referred to as ‘fermentation’, even when there are no coproducts such as ethanol, and even when the process is either aerobic or anaerobic.

One problem with growing green algae at an industrial scale, such as in fermenters with a volume of more than 100 m³, is contamination by other microorganisms.

For instance, industrial-scale fermenters using heterotrophic growth of green algae are often contaminated by bacteria that thrive on sugars, acetate or glycerol. This is described in Jareonsin, Surumpa, and Chayakorn Pumas, “Advantages of heterotrophic microalgae as a host for phytochemicals production”, Frontiers in Bioengineering and Biotechnology 9 (2021): 628597. Jareonsin et al. note that “The major challenge is bacterial contamination in heterotrophic microalgal culture and biomass since the faster grow of bacterial populations is a consequence of commercial applications.”

Other types of contamination by microorganisms that aren't bacteria are contamination by fungi, by yeasts, and by zooplankton. Growth of filamentous fungi can be reduced by turbulence, growth of yeasts can be reduced by elevated pH (above 7.5) and growth of zooplankton such as rotifers can be reduced by CO₂ asphyxiation. What remains to be solved in the art is the biggest problem with contamination when growing green algae, which is bacterial contamination.

Contamination occurs when the growth rate of an undesired microorganism is higher than the growth rate of a desired microorganism. For instance, the doubling time of most lactic acid bacteria is about 0.5 hours and the doubling time of the fastest growing green algae, Chlorella sorokiniana is about 9 hours. This means that over a 24 hour period, a single bacterial cell of lactic acid bacteria grows to 2^{24/0.5} or 3×10¹⁴ cells, whereas a single green algae cell grows to 2^{24/9.0} or 6 cells. Reducing the growth rate of lactic acid bacteria to slightly less than that of green algae (increasing the doubling time to more than 9 hours) completely eliminates the problem of contamination, even over many months of continuous fermentation.

Contamination control is directly related to the time of fermentation, the initial concentration of undesired microorganisms and desired microorganisms, and the growth rate of these organisms. Simple calculations show that longer fermentations have more of a problem with contamination than shorter fermentations, factoring in the different growth rates of these microorganisms.

Therefore, contamination can be controlled by a combination of reducing the fermentation time and reducing the growth rate of contaminating microorganisms (e.g., with antibiotics).

Industrial-scale growth of green algae is necessarily performed in non-aseptic conditions since industrial-scale aseptic growth is prohibitively expensive. The most common methods of contamination control in industrial-scale fermentation of algae have been the use of sulfites (SO₂), antibiotics and peroxides.

Winemakers in ancient Rome burned sulfur candles in wine containers to control contamination that turned wine to vinegar, currently known to be caused by acetic acid bacteria such as Acetobacter aceti. Today, sulfites are often used to prevent bacterial growth in winemaking. However, sulfites cause allergic reactions in some people, and wine labels must have warnings that the wine contains sulfites.

Fuel ethanol producers today often use antibiotics to control bacterial contamination in industrial-scale fermentation. However, this solution is expensive and the resulting food products are contaminated by these antibiotics which enter the food chain, leading to antibiotic resistant bacteria which cause illnesses in people. This is described in Olendorff, Samantha A., Karolina Chmielewska, and Kevin R. Tucker, “Survey of antibiotics residues in DDGS from 14 different states by LCMS”, Cereal Chemistry 98.1 (2021): 81-88.

Attempts have been made to control bacterial contamination using urea hydrogen peroxide and nitrogen-free peroxygen-releasing compounds. The use of urea hydrogen peroxide to control lactobacillus contamination is described in Narendranath, N. V., K. C. Thomas, and W. M. Ingledew, “Urea hydrogen peroxide reduces the numbers of lactobacilli, nourishes yeast, and leaves no residues in the ethanol fermentation”, Applied and Environmental Microbiology 66.10 (2000): 4187-4192. The use of nitrogen-free peroxygen-releasing compounds to control bacterial contamination is described by Solomon in U.S. Pat. No. 8,759,051. However, the use of peroxides to control bacterial contamination has been shown to be uneconomical and is not widely used today in industrial-scale fermentation processes.

The need exists for industrial-scale heterotrophic growth of green algae without contamination by bacteria, without using sulfites, antibiotics or peroxides to control contamination by bacteria.

### SUMMARY OF THE INVENTION

The invention in some variations provides a method for growing green algae, the method comprising growing green algae growth rate for a fermentation time at a fermentation pH in a fermentation broth containing an organic carbon source, urea as a primary nitrogen source, a mineral source, and contaminating bacteria, wherein the green algae belong to the division Chlorophyta, and wherein the amount of nickel in the fermentation broth is maintained below a nickel concentration such that the green algae growth rate is higher than a growth rate of the contaminating bacteria.

In preferred embodiments, the fermentation broth is in operable communication with a heat exchanger, wherein the heat exchanger is a plate heat exchanger comprising titanium heat exchange plates, and wherein the titanium heat exchange plates contain less than 1 g/kg nickel.

In preferred embodiments, the fermentation broth is in operable communication with a heat exchanger, wherein the heat exchanger is a spiral plate heat exchanger comprising titanium heat exchange plates, and wherein the titanium heat exchange plates contain less than 1 g/kg nickel.

In preferred embodiments, the green algae is selected from the group consisting of Chlorella sorokiniana, Chlorella vulgaris, Auxenochlorella protothecoides and combinations thereof.

In preferred embodiments, the organic carbon source is selected from the group consisting of glucose, fructose, mannose, sucrose, maltose, xylose, arabinose, acetate, glycerol, and combinations thereof.

In preferred embodiments, the amount of nickel in the fermentation broth is maintained below 1 mg/kg.

### DETAILED DESCRIPTION OF EMBODIMENTS OF THE INVENTION

The methods, processes, and systems of the present invention will be described in detail by reference to various non-limiting embodiments.

This description will enable one skilled in the art to make and use the invention, and it describes several embodiments, adaptations, variations, alternatives, and uses of the invention. These and other embodiments, features, and advantages of the present invention will become more apparent to those skilled in the art when taken with reference to the following detailed description of the invention.

As used in this specification and the appended claims, the singular forms “a,” “an,” and “the” include plural referents unless the context clearly indicates otherwise. Unless defined otherwise, all technical and scientific terms used herein have the same meaning as is commonly understood by one of ordinary skill in the art to which this invention belongs.

Unless otherwise indicated, all numbers expressing parameters, conditions, results, and so forth used in the specification and claims are to be understood as being modified in all instances by the term “about.” Accordingly, unless indicated to the contrary, the numbers set forth in the following specification and attached claims are approximations that may vary depending upon specific algorithms and calculations.

The term “comprising,” which is synonymous with “including,” “containing,” or “characterized by” is inclusive or open-ended and does not exclude additional, unrecited elements or method steps. “Comprising” is a term of art used in claim language which means that the named claim elements are essential, but other claim elements may be added and still form a construct within the scope of the claim.

As used herein, the phrase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phrase “consisting essentially of” limits the scope of a claim to the specified elements or method steps, plus those that do not materially affect the basis and novel characteristic(s) of the claimed subject matter.

With respect to the terms “comprising,” “consisting of,” and “consisting essentially of,” where one of these three terms is used herein, the presently disclosed and claimed subject matter may include the use of either of the other two terms, except in the case of Markush groups. Thus in some embodiments not otherwise explicitly recited, any instance of “comprising” may be replaced by “consisting of” or, alternatively, by “consisting essentially of.”

No embodiments described herein shall be limited by any theory or speculation regarding reaction mechanisms, mass-transfer mechanisms, or descriptions of feedstocks or products.

The present invention is premised on a technical solution to the serious problem of bacterial contamination during heterotrophic growth of green algae. It has been recognized by the present inventor that bacteria require nickel as a cofactor for urease enzymes in order to use urea for growth while green algae do not require nickel as a cofactor for any enzymes. This principle is applied by designing a fermentation system in which nickel content is minimized in the broth, while at the same time, urea is the primary nitrogen source—which therefore reduces the growth rate of bacteria below the growth rate of green algae, preventing contamination.

Many bacteria, especially many which often contaminate green algae fermentations, do not contain urease enzymes and thus can't grow at all when urea is the nitrogen source. These bacteria include Liquorilactobacillus vini (Lactobacillus vini), Lactobacillus plantarum, Lactobacillus brevis, and Lactobacillus casei. However, two common contaminating bacteria which do contain urease enzymes are Lactobacillus fermentum and Lactobacillus reuteri, and the contamination by these organisms is controlled by reducing the amount of nickel in a fermentation broth.

Reducing the amount of nickel in a fermentation broth has been shown to significantly reduce the growth rate of bacteria in the fermentation broth. Since nickel is a catalyst for urease in bacteria, reducing the concentration of nickel by half reduces the growth of bacteria by half when urea is the primary nitrogen source.

An additional contributor to contamination control is the use of urea as the primary nitrogen source. In this specification, “urea as the primary nitrogen source” means that at least 95%, preferably at least 99%, and most preferably at lease 99.9% (including 100.0%) of the nitrogen atoms taken up by the green algae are derived from urea rather than from other N sources, such as ammonium salts. Lactobacillus fermentum grows slower on urea than ammonium. This is described in Gao, X., S. Y. Qiao, and W. Q. Lu, “Determination of an economical medium for growth of Lactobacillus fermentum using response surface methodology”, Letters in Applied Microbiology 49.5 (2009): 556-561. Many green algae, such as Chlorella sorokiniana, grow faster on urea than other nitrogen sources.

Green algae behave differently than yeast when an excess of urea is in the fermentation broth. When there is an excess of urea, yeast will excrete excess ammonium into the broth while green algae will simply reduce its growth rate. The reason for this is that all yeasts contain one or more of the ATO1, ATO2 and ATO3 enzymes (Ammonia Transport Outward 1,2,3 enzymes, respectively) and no green algae contain these enzymes or even sequence orthologs of these enzymes. This has been confirmed with BLAST searches using uniprot.org.

Extensive searches of the literature were conducted, using scholar.google.com, finding no reports of alkalinization of the fermentation broth when higher levels of urea were used when growing Clorophyta. It is believed that this is due to the absence of ATO1, ATO2 and ATO3 enzymes in Chlorophyta.

Without wishing to be bound by theory, yeasts evolved by growing on carbohydrates and lipids from plants that grow near mammals and the urea from these mammals' urine could produce toxic levels of ammonium when this urea diffused into yeasts. This provided an evolutionary reason for these toxic levels of ammonium to be excreted from yeasts. However, green algae evolved by growing on runoff of mammalian urea from land into bodies of water, and this dilution did not provide any evolutionary reason for ammonium to be excreted from green algae.

The invention in some variations provides a method for growing green algae, the method comprising growing green algae growth rate for a fermentation time at a fermentation pH in a fermentation broth containing an organic carbon source, urea as a primary nitrogen source, a mineral source, and contaminating bacteria, wherein the green algae belong to the division Chlorophyta, and wherein the amount of nickel in the fermentation broth is maintained below a nickel concentration such that the green algae growth rate is higher than a growth rate of the contaminating bacteria.

Green algae has a doubling time ranging from 7 hours to 24 hours, so continuous fermentation is much more cost-effective than a batch fermentation, harvesting about half of the algae every doubling time. Using continuous fermentation in this way, a continuous fermentation of many months can be achieved. Green algae grows at a wide range of pH values, ranging from about pH 4 to pH 8.5. Since yeast grows slowly or not at all at a pH above 7.5, a fermentation pH above 7.5 will eliminate yeast contamination. In some embodiments, the fermentation pH is higher than 7.5, which means at least 7.51. In various embodiments, the fermentation pH is selected as about 7.55, 7.6, 7.7, 7.8, 7.9, 8.0, 8.1, 8.2, 8.3, 8.4, or 8.5, including all intervening ranges. In other embodiments in which yeast contamination is not a concern (but bacteria contamination is a concern), the fermentation pH need not be higher than 7.5.

When urea is the only nitrogen source, bacteria can only grow when there is sufficient nickel in the fermentation broth to catalyze the urease enzyme's conversions of urea to ammonia and CO₂. Green algae instead convert urea to ammonia and CO₂ using the urea carboxylase (EC 6.3.4.6) and allophanate hydrolase (EC 3.5.1.54) enzymes, with biotin as a cofactor (urease uses nickel as a cofactor). This difference between bacteria and green algae is described in Strope, Pooja K., et al., “Molecular evolution of urea amidolyase and urea carboxylase in fungi”, BMC Evolutionary Biology 11.1 (2011): 1-15.

Green algae are biotin prototrophic, so there's no need to add biotin during growth of green algae on urea. This is described in Croft, Martin T., Martin J. Warren, and Alison G. Smith, “Algae need their vitamins”, Eukaryotic cell 5.8 (2006): 1175-1183. Croft et al. show that no Chlorophyta need added biotin to grow using urea.

In acidic solutions containing chloride ions, stainless steel leaches nickel into solution. One way to reduce the amount of nickel in the aqueous fermentation broth is to use heat exchangers made from titanium alloys with trace amounts of nickel. Nickel is a trace element in all titanium alloys except for the nitinol alloy, which has about 50% nickel and 50% titanium.

In preferred embodiments, the fermentation broth is in operable communication (i.e., capable of being heat-exchanged) with a heat exchanger, wherein the heat exchanger is a plate heat exchanger comprising titanium heat-exchange plates, and wherein the titanium heat-exchange plates contain less than 1 g/kg nickel.

In preferred embodiments, the fermentation broth is in operable communication with a heat exchanger, wherein the heat exchanger is a spiral plate heat exchanger comprising titanium heat exchange plates, and wherein the titanium heat exchange plates contain less than 1 g/kg nickel. In this specification, “titanium” includes titanium alloys, provided such alloys contain less than 1 g/kg nickel.

The main source of nickel in a fermenter is from leaching of nickel from the stainless steel in a heat exchanger, and the two most practical heat exchangers for fermenters are wide-gap plate heat exchangers and spiral heat exchangers. These heat exchangers are available commercially using titanium instead of stainless steel and don't leach nickel. The added benefit of using heat exchangers made from titanium alloys is that they aren't subject to corrosion when cooling with sea water, which is an inexhaustible and inexpensive cooling source at an industrial scale.

In preferred embodiments, the green algae is selected from the group consisting of Chlorella sorokiniana, Chlorella vulgaris, Auxenochlorella protothecoides and combinations thereof.

These three green algae are the fastest-growing of the Chlorophyta, and are the widest-cultivated green algae. Chlorella sorokiniana is the fastest growing of these three green algae, and is especially suited to growth using urea as the nitrogen source.

In preferred embodiments, the organic carbon source is selected from the group consisting of glucose, fructose, mannose, sucrose, maltose, xylose, arabinose, acetate, glycerol, and combinations thereof. An organic carbon source excludes CO₂, which is an inorganic carbon source.

Different types of green algae grow on combinations of hexose monomers, hexose dimers, pentose monomers, acetate and glycerol. All green algae grow on glucose, many grow on acetate and glycerol, and only a few grow on sucrose.

In preferred embodiments, the amount of nickel in the fermentation broth is maintained below 1 mg/kg.

When the amount of nickel in the fermentation broth is kept below 1 mg/kg, the growth rate of bacteria is reduced below the growth rate of green algae. There are many techniques for reducing the amount of nickel in the fermentation broth, including coating stainless steel surfaces with inert polymers, using high-density polyethylene as the wall of a fermenter, and using titanium in heat exchangers, for example.

### EXAMPLE

The following example demonstrates the principles of this invention. This invention, as described above, has been shown, by experimental evidence, to be useful for contamination control when growing green algae.

An inoculum of Chlorella vulgaris was prepared by aerobic growth in a nutritional medium comprised of 7 g/L glucose, 1.442 g/L KNO₃, 0.156 g/L KH₂PO₄, 0.1 g/L MgSO₄·7H₂O, 2.6 mg/L FeSO₄·7H₂O, 0.01 g/L Trilon-B (Na₄EDTA), and 8 mg/L CaSO4. After 72 hours of growth, the Chlorella vulgaris was centrifuged and washed several times with distilled water.

In this example, two separate 950 mL glass vessels were each filled with 400 mL of defined media and were each inoculated with equal amounts of Chlorella vulgaris and Lactobacillus fermentum B-8183. Microscopic examination showed roughly equal amounts of Chlorella vulgaris and Lactobacillus fermentum at the start of the fermentation. No stainless steel was in contact with either fermentation broth. A ceramic bubbler was used to oxygenate the fermentation broth with air for 96 hours while the vessels were kept at 34° C. in a dark temperature-controlled cabinet. The organic carbon source in each vessel was 2.8 g of glucose; 0.10 g urea was added to each vessel at the start of fermentation and an additional 0.10 g urea was added every 24 hours. The defined media in both vessels comprised distilled water with 200 mg/L KH₂PO₄, 7 mg/L ZnSO₄, 4 mg/L CuSO₄, 20 mg/L FeSO₄, 5 mg/L MnSO₄, and 100 mg/L MgSO₄. Only one thing was different between vessel 1 and vessel 2—the vessel 2 had 1 mg/L of NiCl₂ added.

Every 24 hours a sample from each vessel was examined under a microscope. In vessel 1, Chlorella vulgaris was about 60%, 70% and 80% of the microorganisms after 24, 48 and 72 hours, respectively. In vessel 2, Lactobacillus fermentum was about 60%, 70% and 90% of the microorganisms after 24, 48 and 72 hours, respectively. This shows that in the absence of nickel, Chlorella vulgaris grew faster than Lactobacillus fermentum, and that in the presence of 1 mg/L of NiCl₂ Lactobacillus fermentum grew faster than Chlorella vulgaris.

At the start of fermentation, the optical density of both vessels was 0.37 and the pH was 6.6. After 24 hours, the optical density of vessel 1 was 0.826 and the optical density of vessel 2 was 1.42, showing that the Lactobacillus fermentum was growing more rapidly in vessel 2. In addition, the pH in vessel 1 rose to 7.1 while the pH in vessel 2 dropped to 5.23, showing that the Lactobacillus fermentum was producing lactic acid in vessel 2 but was not producing lactic acid in vessel 1.

This example clearly demonstrates that when urea is the only nitrogen source and when no sources of nickel are present during fermentation, Chlorella vulgaris (a green algae) grows faster than Lactobacillus fermentum (a bacteria), preventing bacterial contamination.

In this detailed description, reference has been made to multiple specific exemplary embodiments of the invention. These embodiments are described to enable those skilled in the art to practice the invention, and it is to be understood that modifications to the various disclosed embodiments may be made by a skilled artisan.

Where methods and steps described above indicate certain events occurring in certain order, those of ordinary skill in the art will recognize that the ordering of certain steps may be modified and that such modifications are in accordance with the variations of the invention. Additionally, certain steps may be performed concurrently in a parallel process when possible, as well as performed sequentially.

All publications, patents, and patent applications cited in this specification are herein incorporated by reference in their entirety as if each publication, patent, or patent application were specifically and individually put forth herein.

The embodiments and variations described above should provide an indication of the utility and versatility of the present invention. Other embodiments that do not provide all of the features and advantages set forth herein may also be utilized, without departing from the spirit and scope of the present invention. Such modifications and variations are considered to be within the scope of the invention defined by the claims. In the case of conflict in definitions between the present disclosure and a dictionary or other reference, the present disclosure will be controlling.

1. A method for contamination control when growing green algae in the dark, the method comprising heterotrophically growing green algae at a green algae growth rate for a fermentation time at a fermentation pH in a fermentation broth containing an organic carbon source, urea as a primary nitrogen source, a mineral source, said green algae, and contaminating Lactobacillus bacteria, wherein said contaminating Lactobacillus bacteria contain urease enzymes, and wherein said green algae belong to the division Chlorophyta.

2. The method of claim 1, wherein said fermentation broth is in operable communication with a heat exchanger, wherein said heat exchanger is a plate heat exchanger comprising titanium heat exchange plates, and wherein said titanium heat exchange plates contain less than 1 g/kg nickel.

3. The method of claim 1, wherein said fermentation broth is in operable communication with a heat exchanger, wherein said heat exchanger is a spiral plate heat exchanger comprising titanium heat exchange plates, and wherein said titanium heat exchange plates contain less than 1 g/kg nickel.

4. The method of claim 1, wherein said green algae is selected from the group consisting of Chlorella sorokiniana, Chlorella vulgaris, Auxenochlorella protothecoides and combinations thereof.

5. The method of claim 1, wherein said organic carbon source is selected from the group consisting of glucose, fructose, mannose, sucrose, maltose, xylose, arabinose, acetate, glycerol, and combinations thereof.

6. (canceled)
